(19) United States
(12) Patent Application Publication
(10) Pub. No.: US 2018/0333358 A1
(43) Pub. Date: Nov. 22, 2018

(54) DRUG COMPOSITIONS

(71) Applicant: Adamis Pharmaceuticals Corporation, San Diego, CA (US)

(72) Inventor: Dennis J. Carlo, San Diego, CA (US)

(21) Appl. No.: 15/982,958

(22) Filed: May 17, 2018

Related U.S. Application Data
(60) Provisional application No. 62/508,170, filed on May 18, 2017.

(51) Int. Cl. A61K 9/127; A61K 31/728; A61K 31/737; A61P 19/02

(52) U.S. Cl. CPC A61K 9/127; A61P 19/02; A61K 31/737; A61K 31/728

(57) ABSTRACT

Described herein are pharmaceutical compositions for treating a joint disorder. In some embodiments, the compositions can be formulated as liquids for injection and can include a polysulfated xylan and a glycosaminoglycan. Methods of treatment using the compositions are also described.

### CROSS REFERENCE TO RELATED APPLICATIONS

This application claims the benefit of U.S. provisional patent application number 62/508,170, filed May 18, 2017, the entire disclosure of which is incorporated herein by reference.

### FIELD

Described herein are compositions including combination therapy for treating joint disorders.

### SUMMARY

Described herein are pharmaceutical compositions useful for treating and/or preventing joint disorders, conditions/discomfort in mammals. Mammals can include, but are not limited to humans, horses, camels, dogs, cats, cows, bears, rodents, sheep, goats, pigs and the like. In some embodiments, the compositions described herein can be considered veterinary compositions. In some embodiments, the compositions can include a synergistic combination of drugs and/or have an additive drug effect and can be termed combination therapy. In some embodiments, the combination therapy can including a polysulfated xylan and a glycosaminoglycan.

The compositions can include, combined, a polysulfated xylan and a glycosaminoglycan. In one embodiment, the polysulfated xylan is pentosan polysulfate and the glycosaminoglycan is hyaluronic acid.

In some embodiments, combining pentosan polysulfate with hyaluronic acid can provide a surprising synergetic effect that can reduce joint disorder symptoms. In other embodiments, the combination can reduce the amount of each component needed to achieve a similar or better result when compared to the single component.

In some embodiments, combining pentosan polysulfate with hyaluronic acid can provide an additive effect that can reduce joint disorder symptoms.

In some embodiments, the composition is formulated as a non-solid or liquid for injection. In other embodiments, the composition is formulated for oral administration. The non-solid can be a liquid. In other embodiments, the liquid can be formulated for either local injection or systemic injection.

Methods of treating a joint disorder are also described. The methods can comprise administering a composition including the polysulfated xylan and the glycosaminoglycan to a mammal having the joint disorder.

In some embodiments, the administration is performed using an oral syringe.

In some embodiments, the administration is performed using a hypodermic syringe.

In some embodiments, the administration is performed using an intravenous syringe.

In some embodiments, the mammal is a human, a horse, and/or a camel.

Also described are uses a composition for treatment of a joint disorder. The uses can include: administering the composition including a polysulfated xylan encapsulated in a liposome and a glycosaminoglycan to a mammal having the joint disorder, thereby treating the joint disorder.

Also described are uses of a first liquid composition and a second liquid composition for treatment of a joint disorder. The use comprising: administering the first liquid composition including a polysulfated xylan encapsulated in a liposome to a joint having the joint disorder; and administering the second liquid composition including a glycosaminoglycan to the joint having the joint disorder, thereby treating the joint disorder.

Methods of forming a composition including a polysulfated xylan and a glycosaminoglycan are also described. The methods can comprise mixing a combination including the polysulfated xylan and the glycosaminoglycan in a composition for administration. In some embodiments, the mixed composition can also optionally include a preservative and/or a wetting agent.

### DETAILED DESCRIPTION

Described herein are pharmaceutical compositions useful for treating joint disorders in mammals. In some embodiments, the joint disorder can be arthritis including osteoarthritis, rheumatoid arthritis, psoriatic arthritis, juvenille rheumatoid arthritis, gonococcal arthritis, bacterial induced arthritis, reactive arthritis, and/or lyme arthritis, joint inflammation, joint stiffness, interstitial cystitis, bursitis, dislocations, sprains, gout, degenerative joint disease, ankylosing spondylitis, carpal tunnel syndrome, diffuse idiopathic skeletal hyperostosis (DISH), lymes disease, or a combination thereof.

The joint or joints in which the disorder lies can be any joint in a mammal. In some embodiments, the joint can be a pivot joint, a hinge joint, a saddle joint, a planar joint, a condyloid joint, or a ball-and-socket joint. In other embodiments, the joint can be an ankle, shoulder, knee, hip, spinal, elbow, finger, toe, wrist, jaw, or the like. In some embodiments, similar joints may have different terminology between types of mammals and this differing terminology is included herein.

Mammals can include, but are not limited to, humans, horses, camels, dogs, cats, cows, bears, rodents, sheep, goats, pigs and the like.

Treatment of joint disorders can involve pharmaceuticals that have many and/or severe side effects. Prior to discovery of the present compositions, treatment of joint disorders, such as arthritis, can require a patient to take large doses of drugs. In some cases patients take drugs having very severe/serious side effects; in some cases, these drugs are taken in very large doses. Generally doses increase as the disorder progresses. In some circumstances, such as arthritis, no cure exists and the therapies endure indefinitely. Thus, the longer a mammal is subjected to treatment with particular drugs, the more side effects the mammal may be subjected to.

The present combinations can produce synergistic and/or additive effects in reducing a symptom associated with a joint disorder. Consequently, in some embodiments, a reduced, e.g., considerably, dose of therapeutic compounds can be given for an equivalent effect for each individual therapeutic compound. In other embodiments, an equivalent amount of each therapeutic compound, when compared to single compound treatment, can be given to achieve a larger and/or more rapid response. In some embodiments, the compositions can reduce side-effects and drug burden.

As used herein, the term “pharmaceutical composition” refers to a therapeutically effective concentration of the drugs and other ingredients described herein. As used herein, the term “pharmaceutically acceptable” refers to compositions that do not produce an adverse, allergic, or other untoward or unwanted reaction when administered to a mammal.

In some embodiments, the compositions can include a polysulfated xylan in combination with a glycosaminoglycan. In one embodiment, the polysulfated xylan is pentosan polysulfate. In one embodiment, the glycosaminoglycan is hyaluronic acid.

In some embodiments, the polysulfated xylan can be included in the composition at most about 35% (w/v), at most about 40% (w/v), about 15% (w/v), about 20% (w/v), about 25% (w/v), about 30% (w/v), about 35% (w/v), about 40% (w/v), about 45% (w/v), about 50% (w/v), between about 20% and about 40% (w/v), or between about 25% and about 35% (w/v). In some embodiments, the glycosaminoglycan can be included in the composition at most about 30% (w/v), at most about 25% (w/v), about 10% (w/v), about 15% (w/v), about 20% (w/v), about 21% (w/v), about 22% (w/v), about 23% (w/v), about 25% (w/v), about 30% (w/v), about 35% (w/v), about 40% (w/v),between about 20% and about 25% (w/v), or between about 15% and about 30% (w/v).

In some embodiments, the polysulfated xylan can be delivered in particle weights per dose. Weights can include between about 10 mg and about 1,000 mg, between about 10 mg and about 100 mg, between about 10 mg and about 500 mg, between about 10 mg and about 50 mg, between about 10 mg and about 400 mg, between about 10 mg and about 300 mg, between about 10 mg and about 200 mg, between about 100 mg and about 400 mg, between about 100 mg and about 500 mg, or between about 1 mg and about 1,000 mg.

The drug combination or combination therapy may be made into a liquid formulation. Liquid formulations suitable for enteral or parenteral administration include, without limitation, solutions, syrups, elixirs, dispersions, emulsions, and suspensions. The drug combination or combination therapy disclosed herein intended for such administration may be prepared according to any method known in the art for the manufacture of pharmaceutical compositions. In such liquid dosage forms, the drug combination disclosed herein may be admixed with (a) suitable aqueous and non-aqueous carriers, (b) diluents, (c) solvents, such as, e.g., water, ethanol, propylene glycol, polyethyleneglycol, glycerol, vegetable oils, such as, e.g., rapeseed oil and olive oil, and injectable organic esters such as ethyl oleate; and/or fluidity agents, such as, e.g., surfactants or coating agents like lecithin. In the case of dispersions and suspensions, fluidity can also be controlled by maintaining a particular particle size.

The drug combination or combination therapy disclosed herein may be made into a semi-solid formulation. Semi-solid formulations can be made for enteral or topical administration. Semi-solid formulations suitable for enteral administration include, without limitation, pastes, and gels. Semi-solid formulations suitable for topical or oral administration include, without limitation, ointments, creams, salves, pastes, and gels. The drug combination or combination therapy disclosed herein intended for such administration may be prepared according to any method known in the art for the manufacture of pharmaceutical compositions.

Liquid and/or semi-solid formulations may be formulated with sweetening agents, for example glycerol, propylene glycol, sorbitol or sucrose. Such formulations may also contain one or more demulcent, preservative, flavoring agent, and/or coloring agent.

Liquid and/or semi-solid suspensions may be formulated by suspending the drug combination in an admixture with suitable excipients. Suitable excipients can be suspending agents, such as sodium carboxymethyl cellulose, methylcellulose, hydroxypropylmethylcellulose, sodium alginate, pectin, polyvinyl pyrrolidone, polyvinyl alcohol, natural gum, agar, gum tragacanth and gum acacia.

Oily suspensions may be formulated by suspending the drug combination in an admixture with (a) vegetable oils including almond oil, arachis oil, avocado oil, canola oil, castor oil, coconut oil, corn oil, cottonseed oil, grape seed oil, hazelnut oil, hemp oil, linseed oil, olive oil, palm oil, peanut oil, rapeseed oil, rice bran oil, safflower oil, sesame oil, soybean oil, soya oil, sunflower oil, walnut oil, wheat germ oil, or a combination thereof, (b) a saturated fatty acid, an unsaturated fatty acid, or a combination thereof, such as palmitic acid, stearic acid, oleic acid, linoleic acid, linolenic acid, or a combination thereof, (c) mineral oil such as liquid paraffin, (d) surfactants or detergents. The oily suspensions may contain a thickening agent such as beeswax, hard paraffin, or cetyl alcohol.

In some embodiments, the liquid or semi solid formulations can include liposomes. More specifically, liposomes can be loaded with drugs. Liposomes include phospoholipid bilayers and can be formulated with various diameters. In some embodiments, the liposomes can be micrometer(s) in size. In other embodiments, the liposomes can be nanometer(s) in size.

In some embodiments, the liposomes can mimic cell walls and/or protect the drug that it encases. In some embodiments, liposomes can fuse with a cell wall and allow a drug(s) to be delivered into the cell.

Liposomes can be used to encapsulate the polysulfated xylan. In other embodiments, the liposomes can encapsulate the glycosaminoglycan. In still other embodiments, liposomes can encapsulate both the polysulfated xylan and the glycosaminoglycan in the same liposome.

In some embodiments, the liposomes can be between about 50 nm and about 200 nm, between about 100 nm and about 500 nm, between about 500 nm and about 1,000 nm, between about 1,000 nm and about 5,000 nm, between about 1 nm and about 500 nm, between about 10 nm and about 100 nm, between about 10 nm and about 250 nm, between about 10 nm and about 1,000 nm, between about 10 nm and about 1,000 nm, or between about 10 nm and about 5,000 nm in diameter.

A composition disclosed herein may optionally include a pharmaceutically-acceptable carrier that facilitates processing of drug(s) into pharmaceutically-acceptable compositions. Such a carrier can be mixed with a drug or drugs or permitted to dilute or enclose the drug or drugs and can be a solid, semi-solid, or liquid agent. It is understood that the drug or drugs can be soluble or can be delivered as a suspension in the desired carrier or diluent. Any of a variety of pharmaceutically acceptable carriers can be used including, without limitation, aqueous media such as, e.g., water, saline, glycine, and the like; solid carriers such as, e.g., polyethylene glycol, polyethylene oxide, mannitol, lactose, starch, magnesium stearate, sodium saccharin, talcum, cellulose, glucose, sucrose, magnesium carbonate, and the like; solvents; dispersion media; coatings; isotonic and absorption delaying agents; or any other inactive ingredient. Selection of a pharmacologically acceptable carrier can depend on the mode of administration.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, flavoring agents, coloring agents, suspension agents, and the like.

Various buffers and means for adjusting pH can be used to prepare a pharmaceutical composition disclosed herein. Such buffers include, without limitation, acetate buffers, citrate buffers, phosphate buffers, neutral buffered saline, phosphate buffered saline, bicarbonate buffers, and borate buffers. It is understood that acids or bases can be used to adjust the pH of a composition as needed. Bases can include sodium hydroxide. In one embodiment, sodium bicarbonate is used as a buffer. In another embodiment, sodium hydroxide is used to make the composition more alkaline. In some embodiments, the alkalinity of the composition can be a pH greater than about 7, greater than about 7.5, greater than about 8, greater than about 8.5, greater than about 9, greater than about 9.5, or greater than about 10

In some embodiments, a buffer(s) can be included in the composition at most about 10% (w/v), at most about 5% (w/v), about 1% (w/v), about 2% (w/v), about 3% (w/v), about 4% (w/v), about 5% (w/v), about 6% (w/v), about 7% (w/v), about 8% (w/v), about 9% (w/v), about 10% (w/v), between about 1% and about 10% (w/v), or between about 4% and about 6% (w/v).

Pharmaceutically acceptable antioxidants can include, without limitation, sodium metabisulfite, sodium thiosulfate, acetylcysteine, butylated hydroxyanisole and butylated hydroxytoluene.

If formulated for oral delivery, flavoring agents can provide a composition that smells good and/or tastes good. Non-human mammals, and even humans in same cases, may need encouragement to consume the compositions described. Thus, flavoring agents may be added to stimulate appeal or naturally attract a particular mammal species. Flavoring agents can make orally administrable compositions taste like apple, orange, lemon, grape, butterscotch, cherry, blueberry, raspberry, strawberry, honey, peppermint, spearmint, cinnamon, peach, watermelon, chocolate, espresso, mango, banana, carrot, cantaloupe, guava, acai, cheese, tomato, caramel, taffy, lime, and the like. Flavor combinations can also be provided.

Preservatives can include, without limitation, sodium sulfite, sodium sulfide, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. In one embodiment, sodium sulfite is used as a preservative.

In some embodiments, a preservative(s) can be included in the composition at most about 0.2% (w/v), at most about 0.5% (w/v), about 0.05% (w/v), about 0.06% (w/v), about 0.07% (w/v), about 0.08% (w/v), about 0.09% (w/v), about 0.1% (w/v), about 0.11% (w/v), about 0.12% (w/v), about 0.13% (w/v), about 0.14% (w/v), about 0.15% (w/v), between about 0.05% and about 0.15% (w/v), or between about 0.09% and about 0.11% (w/v).

Tonicity adjustors can include, without limitation, salts such as, e.g., sodium chloride, potassium chloride, mannitol or glycerin and other pharmaceutically acceptable tonicity adjustors.

Suspension agents can include, without limitation, carboxymethyl cellulose. The suspension agent can be used to keep the drug(s) dispersed evenly throughout the composition.

In some embodiments, the suspension agent(s) can be included in the composition at most about 0.2% (w/v), at most about 0.5% (w/v), about 0.05% (w/v), about 0.1% (w/v), about 0.2% (w/v), about 0.3% (w/v), about 0.4% (w/v), about 0.5% (w/v), about 0.8% (w/v), about 1% (w/v), between about 0.1% and about 0.3% (w/v), or between about 0.1% and about 1% (w/v).

In other embodiments, the drug combination disclosed herein may be made into an inhaled formulation. Inhaled formulations suitable for enteral or parenteral administration include, without limitation, aerosols, and dry powders. The drug combination disclosed herein intended for such administration may be prepared according to any method known in the art for the inhalable manufacture of pharmaceutical compositions.

The drug combination may be made into a solid formulation. Solid formulations suitable for enteral or parenteral administration include, without limitation, capsules, tablets, pills, troches, lozenges, powders and granules suitable for inhalation or for reconstitution into sterile injectable solutions or dispersions. The drug combination intended for such administration may be prepared according to any method known to the art for the manufacture of pharmaceutical compositions. In such solid dosage forms, the drug combination may be admixed with (a) at least one inert customary excipient (or carrier), such as, e.g., sodium citrate or dicalcium phosphate or (b) fillers or extenders, as for example, starch, lactose, sucrose, glucose, mannitol, isomalt, and silicic acid, (c) binders, such as, e.g., carboxymethylcellulose, alignates, gelatin, polyvinylpyrrolidone, sucrose and acacia, (d) humectants, such as, e.g., glycerol, (e) disintegrating agents, such as, e.g., agar-agar, calcium carbonate, corn starch, potato starch, tapioca starch, alginic acid, certain complex silicates and sodium carbonate, (f) solution retarders, such as, e.g., paraffin, (g) absorption accelerators, such as, e.g., quaternary ammonium compounds, (h) wetting agents, such as, e.g., cetyl alcohol and glycerol monostearate, (i) adsorbents, such as, e.g., kaolin and bentonite, (j) lubricants, such as, e.g., talc, stearic acid, calcium stearate, magnesium stearate, solid polyethylene glycols, sodium lauryl sulfate or mixtures thereof, and (k) buffering agents. The tablets may be uncoated or they may be coated by known techniques to delay disintegration and absorption in the gastrointestinal tract and thereby provide a sustained action over a longer period.

In some embodiments, the polysulfated xylan is loaded into a liposome as described herein for delivery. In other embodiments, the pentosan polysulfate is loaded into a liposome as described herein for delivery. In still other embodiments, liposomes can encapsulate both the polysulfated xylan and the glycosaminoglycan in the same liposome for delivery.

Combining pentosan polysulfate with hyaluronic acid can provide a surprising synergistic effect that can reduce symptoms associated with joint disorders. In other embodiments, combining pentosan polysulfate with hyaluronic acid can provide a surprising synergistic effect that can reduce pain associated with joint disorders. In other embodiments, combining pentosan polysulfate with hyaluronic acid can provide a surprising synergistic effect that can reduce inflammation associated with joint disorders.

The compositions disclosed herein may be formulated for either local or systemic delivery.

In some embodiments, a composition disclosed herein can be provided for injection, e.g., local injection. The composition can be injected directly into a joint(s) affected by the joint disorder. In other embodiments, the compositions can be injected adjacent to or near the affected joint(s). In still other embodiments, the compositions can be injected systemically. In other embodiments, combinations of injections paradigms can be utilized.

In some embodiments, the polysulfated xylan can be injected in an affected joint while the glycosaminoglycan is delivered systemically. In other embodiments, the glycosaminoglycan can be injected in an affected joint while the polysulfated xylan is delivered systemically. In still other embodiments, both the polysulfated xylan and the glycosaminoglycan can be injected in an affected joint. In some embodiments, the polysulfated xylan and the glycosaminoglycan can be injected together in an single injection or can be injected separately.

The compositions described herein can be administered at amounts of about 10 cc, about 15 cc, about 20 cc, about 25 cc, about 30 cc, about 35 cc, about 40 cc, about 45 cc, about 50 cc, about 55 cc, about 60 cc, about 65 cc, about 70 cc, about 75 cc, about 80 cc, about 85 cc, about 90 cc, about 95 cc, about 100 cc, at least about 10 cc, at least about 20 cc, between about 20 cc and about 50 cc, between about 20 cc and about 30 cc, between about 25 cc and about 35 cc, between about 30 cc and about 35 cc, or between about 25 cc and about 30 cc per administration.

Administration can be any interval that results in a therapeutic response. In some embodiments, administration can be one, two, three, four, five, or more times per day. In other embodiments, administration can be every other day, every third day, every fourth day, every fifth day, every sixth day, once per week, twice per month, monthly, and the like. In one embodiment, administration is once per day.

Methods of forming a composition including a polysulfated xylan and a glycosaminoglycan are also described. The methods can comprise mixing a combination including the polysulfated xylan and the glycosaminoglycan in a composition for administration. In some embodiments, the mixed composition can also optionally include a preservative.

Some embodiments provide a kit including a syringe pre-filled with a composition including polysulfated xylan and a glycosaminoglycan described herein and instructions for use in a unifying container.

Some embodiments provide a kit including a first syringe pre-filled with a composition including polysulfated xylan, a second syringe pre-filled with a composition including a glycosaminoglycan, and instructions for use in a unifying container.

Other embodiments provide a kit including a vial or other appropriate container containing a composition described herein and instruction for use in a unifying container. Still other embodiments provide a kit including a first and second vial or other appropriate container containing a first composition including polysulfated xylan and a second composition including a glycosaminoglycan respectively and instruction for use in a unifying container.

Still other embodiments provide a kit including a vial or other appropriate container containing a composition described herein, a syringe, and instruction for use in a unifying container. Other embodiments provide a kit including a first and second vial or other appropriate container containing a first composition including polysulfated xylan and a second composition including a glycosaminoglycan respectively, two syringes, and instruction for use in a unifying container.

Some embodiments provide a kit including any number of syringes pre-filled with a composition or two compositions described herein and instruction for a scheduled use in a unifying container.

Other embodiments provide a kit including a vial(s) or other appropriate container containing a composition described herein for a scheduled use and instruction for use in a unifying container.

Once housed in a delivery device or included in a kit, the composition/delivery devices/kits can be sterilized using conventional sterilization techniques without substantial degradation to the composition. Without substantial degradation to the composition means that the composition retains greater than 80%, greater than 90%, greater than 95%, or greater than 99% of its activity. In some embodiments, the compositions remain unaffected by sterilization. Sterilization can be by at least one sterilization technique including, but not limited to gamma irradiation, pressure sterilization and/or steam sterilization.

In some embodiments, the compositions described herein can retain substantially all potency for at least 14 days. In some embodiments, the compositions described herein can retain substantially all potency for at least 30 days. In some embodiments, the compositions described herein can retain substantially all potency for at least 60 days. In some embodiments, the compositions described herein can retain substantially all potency for at least 90 days. In some embodiments, the compositions described herein can retain substantially all potency for at least 180 days. In some embodiments, the compositions described herein can retain substantially all potency for at least 360 days. In some embodiments, the compositions described herein can retain substantially all potency for longer than 360 days.

Substantially all potency means that the composition(s) retains at least greater than 80%, greater than 90%, greater than 95%, or greater than 99% of its activity when stored at appropriate conditions. In some embodiments, appropriate conditions can be a room temperature. In some embodiments, appropriate conditions can be without direct light. In some embodiments, appropriate conditions can be refrigerated.

The compositions described herein can reduce the incidence symptoms of a joint disorder. In one embodiment, the compositions described herein can reduce the incidence symptoms of arthritis. In one embodiment, the compositions described herein can reduce the incidence symptoms of osteoarthritis. In some embodiments, the compositions can simply treat a joint disorder.

In some embodiments, the herein described compositions can reduce joint disorder symptoms when compared to treatment with a polysulfated xylan alone by at least about 10%, by at least about 20%, by at least about 50%, by at least about 75%, by at least about 100%, by at least about 150%, by at least about 200%.

In some embodiments, the herein described compositions can reduce joint disorder symptoms when compared to treatment with a glycosaminoglycan alone by at least about 10%, by at least about 20%, by at least about 50%, by at least about 75%, by at least about 100%, by at least about 150%, by at least about 200%.

In some embodiments, the herein described compositions can reduce joint disorder symptoms when compared to treatment with pentosan polysulfate alone by at least about 10%, by at least about 20%, by at least about 50%, by at least about 75%, by at least about 100%, by at least about 150%, by at least about 200%.

In some embodiments, the herein described compositions can reduce joint disorder symptoms when compared to treatment with hyaluronic acid alone by at least about 10%, by at least about 20%, by at least about 50%, by at least about 75%, by at least about 100%, by at least about 150%, by at least about 200%.

In one embodiment, as a result of the reduced symptoms associated with joint disorders, a composition disclosed herein may reduce/remove/alleviate an unwanted side effect elicited by administration of a polysulfated xylan and/or a glycosaminoglycan. In other embodiments, as a result of the reduced symptoms associated with joint disorders, a composition disclosed herein may reduce an unwanted side effect elicited by administration of pentosan polysulfate and/or hyaluronic acid.

In one embodiment, unwanted side effects of pentosan polysulfatinclude can include without limitation diarrhea, heartburn, stomach pain, hair loss, headache, rash, insomnia, bruising more easily, excess bleeding, and the like. In one embodiment, unwanted side effects of hyaluronic acid include, without limitation, constipation, diarrhea, injection site pain, mild rectal bleeding, mild rectal itching or discomfort, dizziness, chills, cold sweat, stomach pain, or pain during sexual intercourse.

Further, as a result of the synergistic and/or additive effects of the herein described compositions, a reduced load of drugs may be required to achieve similar results to the drugs alone. In some embodiments, treatment using the described compositions may only require about 10%, about 20%, about 30%, about 40%, about 50%, about 60%, about 70%, or about 80% of a general dose of pentosan polysulfate. In other embodiments, treatment using the described compositions may only require about 10%, about 20%, about 30%, about 40%, about 50%, about 60%, about 70%, or about 80% of a general dose of hyaluronic acid.

In some embodiments, compositions described herein can be administered regularly to aid in joint disorder symptoms before they occur. The compositions can be administered to prevent recurrence of the joint disorder symptoms. In other embodiments, the compositions can be administered before evidence of symptoms to prevent them.

### EXAMPLE 1

### Knee Treatment

An arthritic horse is treated with a composition including pentosan polysulfate and hyaluronic acid formulated within liposomes. The horse has arthritis in its front right knee. The composition is injected into the knee. The horse presents a reduction in pain when walking on the right leg.

The horse is given injections in the affected knee once a week to reduce the arthritic symptoms.

### EXAMPLE 2

### Hip Treatment

An arthritic patient is injected in his affected hip with a first composition including pentosan polysulfate. He is also injected in the same affected hip with a second composition including hyaluronic acid. The patient presents a reduction in pain when walking using the hip joint.

The patient is given injections in the affected hip once a week to reduce the arthritic symptoms.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A composition including:
a polysulfated xylan encapsulated in a liposome, and
a glycosaminoglycan,
wherein the composition is formulated as a liquid for administration to a joint.

2. The composition of claim 1, wherein the polysulfated xylan is pentosan polysulfate.

3. The composition of claim 1, wherein the polysulfated xylan is present at between about 50 mg and about 200 mg.

4. The composition of claim 1, wherein the glycosaminoglycan is hyaluronic acid.

5. The composition of claim 1, wherein the glycosaminoglycan is present at a concentration of between about 10% w/v and about 50% w/v.

6. The composition of claim 1 formulated as a liquid.

7. A treatment composition including:
a first liquid including polysulfated xylan encapsulated in a liposome, and
a second liquid including a glycosaminoglycan,
wherein the composition is formulated as a liquid for administration to a joint.

8. The composition of claim 7, wherein the polysulfated xylan is pentosan polysulfate.

9. The composition of claim 7, wherein the polysulfated xylan is present at between about 50 mg and about 200 mg.

10. The composition of claim 7, wherein the glycosaminoglycan is hyaluronic acid.

11. The composition of claim 7, wherein the glycosaminoglycan is present at a concentration of between about 10% w/v and about 50% w/v.

12. A method of treating a joint disorder, the method comprising:
administering a composition including a polysulfated xylan encapsulated in a liposome and a glycosaminoglycan to a mammal having the joint disorder, and
treating the joint disorder.

13. The method of claim 12, wherein the polysulfated xylan is pentosan polysulfate.

14. The method of claim 12, wherein the polysulfated xylan is present at between about 50 mg and about 200 mg.

15. The method of claim 12, wherein the glycosaminoglycan is hyaluronic acid.

16. The method of claim 12, wherein the glycosaminoglycan is present at a concentration of between about 10% w/v and about 50% w/v.

17. The method of claim 12, wherein the administering is by injection into the joint.

18. A method of treating a joint disorder, the method comprising:
administering a first liquid composition including a polysulfated xylan encapsulated in a liposome to a joint having the joint disorder; and
administering a second liquid composition including a glycosaminoglycan to the joint having the joint disorder, and treating the joint disorder.

19. The method of claim 18, wherein the polysulfated xylan is pentosan polysulfate.

20. The method of claim 18, wherein the polysulfated xylan is present at between about 50 mg and about 200 mg.

21. The method of claim 18, wherein the glycosaminoglycan is hyaluronic acid.

22. The method of claim 18, wherein the glycosaminoglycan is present at a concentration of between about 10% w/v and about 50% w/v.

23. The method of claim 18, wherein the administering is by injection into the joint.
